(19) United States
(12) Patent Application Publication
(10) Pub. No.: US 2012/0022114 A1
(43) Pub. Date: Jan. 26, 2012

(54) Low-dose, non-irritating nicotine nasal composition to reduce the desire to smoke

(75) Inventors: Jorge Alberto Cassara, Buenos Aires (AR); Julio Cesar Vega, Buenos Aires (AR)

(73) Assignee: Laboratorio Pablo Cassara S.R.L., Buenos Aires (AR)

(21) Appl. No.: 13/253,047

(22) Filed: Oct. 4, 2011

Related U.S. Application Data
(63) Continuation of application No. 12/550,355, filed on Aug. 29, 2009, now abandoned.

(51) Int. Cl. A61K 31/4439; A61P 25/34

(52) U.S. Cl. 514/343

(57) ABSTRACT

A composition for administration to the nasal mucosa of a subject comprises a solution of nicotine or a pharmaceutically acceptable salt thereof in a pharmaceutically acceptable solvent. The composition has a nicotine concentration less than 0.08 mg/ml. The composition used alone assists in reduction of the desire of a subject to smoke tobacco. It also reduces the nasal symptoms associated with administration of higher concentrations of nicotine to the nasal mucosa.

This invention relates to compositions and methods useful for human subjects who wish to reduce tobacco smoking without experiencing any discomfort associated with the use of available methods.

### BACKGROUND OF THE INVENTION

Both the increasing conscience of the harmful effects of tobacco smoking and the increasing delineation of smoke-free public areas have put greater pressure on tobacco smokers to either quit smoking or find alternative methods to reduce the desire to smoke leading ultimately to cessation of smoking Various forms of nicotine-replacement therapies have been suggested and these include:

Nicotine-containing chewing gum is available commercially and has provided a satisfactory substitute for tobacco smoking for some people. However, some people cannot adhere to this treatment because of the unpleasant side effects (nausea and indigestion) and taste (Jarvis et al., British Medical Journal, Vol. 285, p. 537 (1982); Schneider, Comprehensive Therapy, Vol. 13, p. 32 (1987).

Nicotine-containing nose drops have been reported (Russel et al., British Medical Journal, Vol. 286, p. 683 (1983); Jarvis et al., Brit. J. of Addiction, Vol. 82, p. 983 (1987)). Nose drops, however, are difficult to administer and are not convenient for use at work or in other public situations.

Skin patches for transdermal administration of nicotine are also available commercially,

U.S. Pat. Nos. 4,920,989 and 4,953,572 disclose the use of a nicotine inhalation aerosol, sometimes in conjunction with nicotine skin patches, as a means of reducing tobacco smoking Although a certain degree of airway irritation is desired to mimic smoking, this cannot be readily controlled and the irritation may be pronounced, making the use of a nicotine aerosol undesirable. Although a certain degree of airway irritation is desired to mimic smoking, this cannot be readily controlled and the irritation may be pronounced, making the use of a nicotine aerosol undesirable.

Perkins et al. (Behavior, Research Methods, Instruments and Computers (1986), vol. 18, p. 420 and Psychopharm. (1989), vol. 97, p. 529) reported use of a nicotine aerosol spray administered slowly in a total duration of 5 minutes. This administration is not practical for use in public places.

U.S. Pat. No. 4,579,858 discloses a nicotine-containing preparation of high concentration and viscosity, which is administered to the nose as a viscous plug.

U.S. Pat. No. 5,656,255 discloses a nicotine nasal spray having a concentration of 10 to 40 mg/ml. A commercial nicotine nasal spray containing a solution of nicotine in this range is already commercially available and this product produces a very strong irritation of the nasal mucosa resulting in production of excessive tears and runny nose, besides the physical discomfort. Even though this effect gets milder after using the nasal spray for prolonged time, several people find this unpleasantness unacceptable resulting in lack of compliance.

The U.S. Pat. No. 6,596,740 discloses a nasal spray of nicotine having a concentration above 1.0 mg/ml.

In conclusion, therefore there remains a need for a nicotine preparation suitable to eliminate the craving to smoke, which can be conveniently used in public or out of home and which is pleasant and quick enough to effectively and immediately prevent people from smoking

### SUMMARY OF THE INVENTION

In the present invention, a diluted aqueous solution of nicotine less than 0.08 mg/mL or a pharmaceutically acceptable salt thereof, with the addition of adequate excipients, packaged into a bottle fitted with a metering pump suitable for nasal administration can effectively and surprisingly eliminate the craving to smoke either to avoid smoking in public places or helping smokers to stop smoking The application of one shot in each nasal cavity takes just seconds without any adverse effects.

### DETAILED DESCRIPTION OF THE INVENTION

The present invention provides a convenient, inexpensive, non-irritating and effective alternative to tobacco smoking, by administration of a very low dose of nicotine by nasal spray to a subject.

This smoking alternative may be used both for trying to quit tobacco smoking or for refraining from smoking in public places, thus avoiding the undesired effects of tobacco smoking on people near the smoker and the damages caused by other substances present in tobacco like carcinogens and carbon monoxide.

Jones teaches in U.S. Pat. No. 5,656,255 that the mechanism of action of nicotine administration is directly related with systemic blood levels of nicotine. He thus concludes that the efficacy of the nicotine nasal spray disclosed there to reduce craving to smoke is related to this parameter. However, in the present invention, it was discovered that surprisingly a very dilute nicotine solution with the appropriate composition could be effective in eliminating the craving to smoke even in heavy smokers. The present invention has the additional benefit that no irritation is caused to the nasal mucosa and the smoker can use this spray in public places without going through the unpleasant sensations of itching, stinging, tearing and having a runny nose for several minutes after its application. The lack of common side effects also helps maintain compliance of users. Therefore the Jones U.S. Pat. No. 5,656,255 is not a prior art for the present invention.

The Jones U.S. Pat. No. 6,596,740 is also not a prior art for this invention; it is noteworthy that Jones teaches a nasal spray of 10 micron or more in size and a concentration of 1 to less than 9 mg/ml. Fact that Jones does not extend the range of concentration to below 1 mg/ml is significant and not to be considered obvious to use a concentration below 1 mg/ml; the action of nicotine is based on achieving a certain blood concentration and thus the required concentration in the case of Jones, with a particular droplet size to perhaps improve impaction is required. The present invention does not require any specific droplet size and reports a concentration that might have been considered ineffective by Jones.

The Baker et al, U.S. Pat. No. 5,721,257 is also not a prior art for the present invention. Baker recommends using less than 1 mg/ml concentration of nicotine and while it may appear that the limit of 0.08 mg/ml as reported in the specification would constitute prior art but Baker claims a combination of treatments, not a single treatment as the requirement of invention, wherein the first significant step Baker's invention is a transdermal treatment to obtain nicotine levels of between 5 and 35 ng/ml for at least 12 hours and then starting the second treatment that comprises administering nicotine by transmucosal route. Baker never proposes to use the transmucosal route alone, as provided in the present invention. This would have been unanticipated in light of the requirement of obtaining a certain blood concentration of nicotine; in the case of Baker, the transmucosal route is merely providing a sustaining function to the treatment achieved by transdermal route. Taken out of context, the recommendation by Baker of the concentration of 0.1 to 100 mg/ml is redundant for the present invention as there is no plausible or obvious way to predict that a concentration reported in the present invention of less than 0.08 mg/ml would be effective given alone. The present invention also restricts the amount of nicotine administered by specifying a dose quantity of 25 to 100 ul; it is unlikely that any significant blood concentration would be achieved at this low dose level and in all likelihood, without claiming any specific mechanism involved here, a different mechanism of action is operational which could not be predicted or be obvious based on Baker and Jones.

In the present invention, nicotine or a pharmaceutically acceptable salt thereof is dissolved in water with the addition of pharmaceutically acceptable salts in a slightly acidic to neutral pH range. Optionally, the invention may be fortified with a viscosity-increasing agent such as a cellulose derivative or other pharmaceutically acceptable polymer or agent of other kind The invention is a solution, which is packaged into a bottle fitted with a metering pump suitable for nasal administration. Both usual metering pumps and preservative-free metering pumps can be used for the invention. In the former case, preservatives should be added to the nicotine solution to avoid microbial contamination. In the latter case, nicotine solution should be sterilized by filtration and filled aseptically into the bottles fitting the pumps on them also under aseptic conditions.

Pharmaceutically acceptable salts of nicotine are nicotine tartratre, nicotine hydrogen tartrate, nicotine Citrate, nicotine hydrogen citrate, nicotine dihydrogen Citrate and others known to those skilled in the art.

Buffering agents could be selected from buffers composed of Phosphates, Sodium Citrates or other well known to those skilled in the art.

Sodium chloride, mannitol, xylitol or other suitable substances soluble in water can be added to regulate the osmotic pressure of the preparation and achieve isotonicity.

Viscosity—increasing agents could be derivatives of cellulose, povidone, bentonitas or other well known to those skilled in the art.

Flavors, such as menthol or others known to the skilled in the art could be added.

If a usual metering pump is employed, suitable preservatives should be added, such as benzalkonium chloride, benzoic acid, or others well known to the skilled in the art.

The inventors found that a volume between 25 and 100 μl can be adequate for the administration of nicotine solution.

As nicotine enters the body, it is distributed quickly through the bloodstream and can cross the blood-brain barrier. On average it takes about seven seconds for the substance to reach the brain when inhaled. The half-life of nicotine in the body is around two hours. The amount of nicotine absorbed by the body from smoking depends on many factors, including the type of tobacco, whether the smoke is inhaled, and whether a filter is used. For chewing tobacco, dipping tobacco, snus and snuff, which are held in the mouth between the lip and gum, or taken in the nose, the amount released into the body tends to be much greater than smoked tobacco. Nicotine acts on the nicotinic acetylcholine receptors, specifically the ganglion type nicotinic receptor and one CNS nicotinic receptor. The former is present in the adrenal medulla and elsewhere, while the latter is present in the central nervous system (CNS). In small concentrations, nicotine increases the activity of these receptors. Nicotine also has effects on a variety of other neurotransmitters through less direct mechanisms. By binding to nicotinic acetylcholine receptors, nicotine increases the levels of several neurotransmitters—acting as a sort of “volume control.” It is thought that increased levels of dopamine in the reward circuits of the brain are responsible for the euphoria and relaxation and eventual addiction caused by nicotine consumption. A single amino-acid difference between brain and muscle acetylcholine receptors explains why nicotine activates the CNS but does not activate skeletal muscles and cause present death. Nicotine addiction is therefore a biological fluke.

Tobacco smoke contains the monoamine oxidase inhibitors harman, norharman, anabasine, anatabine, and nornicotine. These compounds significantly decrease MAO activity in smokers. MAO enzymes break down monoaminergic neurotransmitters such as dopamine, norepinephrine, and serotonin. Chronic nicotine exposure via tobacco smoking up-regulates alpha-4-beta-2-nAChR in cerebellum and brainstem regions but not habenulopeduncular structures. Alpha-4-beta-2 and alpha-6-beta-2 receptors, present in the ventral tegmental area, play a crucial role in mediating the reinforcement effects of nicotine.

Nicotine also activates the sympathetic nervous system, acting via splanchnic nerves to the adrenal medulla, stimulates the release of epinephrine. Acetylcholine released by preganglionic sympathetic fibers of these nerves acts on nicotinic acetylcholine receptors, causing the release of epinephrine (and norepinephrine) into the bloodstream.

Surprisingly the use of a nasal spray according to this invention is extremely well tolerated with no side effects, not even temporary nasal irritation or runny nose. Inventors hypothesize, though such hypothesis is not needed for establishing the invention, that this surprising result could be related to nose-to-brain mechanism, i.e., very small doses of nicotine could go directly from the nose into the brain and be enough to eliminate the desire to smoke. This invention also raises a question about the blood-level and activity relationships of the effect of nicotine that have long been purported and as a result all previous inventions have attempted to use higher concentrations to achieve certain pre-defined blood levels. In some instances, inventors have tried to control the particle size to improve absorption and thus the blood level while all such attempts are considered futile given the unique mechanism of action observed in the present invention.

### EXAMPLE 1

The following composition is prepared:

TABLE 1

| Component | Amount | Unit |
|---|---|---|
| Nicotine | 8.0 | mg |
| Disodium EDTA | 1.00 | mg |
| Citric Acid anhydrous | 2.86 | mg |
| Disodium Phosphate anhydrous | 75.4 | mg |
| Monosodium Phosphate anhydrous | 57.0 | mg |
| Methyl Paraben | 32.50 | mg |
| Propyl Paraben | 7.50 | mg |
| Sodium Chloride | 840.0 | mg |
| Polysorbate 80 | 500.0 | mg |
| Flavor | 200.0 | mg |
| Purified Water, qs | 100 | ml |

The above solution is filled into bottles and metering pumps are fitted on them.

### EXAMPLE 2

A composition exactly like the one depicted in table 1 but without the addition of preservatives, i.e., without methyl- and propylparaben. This composition can be prepared in an analogous way to the one described in Example 1, but the primary package should be a bottle fitted with a metering pump suitable for preservative free formulations.

### EXAMPLE 3

These nasal sprays were distributed to 12 heavy smokers (i.e., subjects smoking more than one pack of cigarettes a day). They were instructed to spray at least two shots into the air away from their faces before starting to use them and to spray one shot into each nostril whenever they felt craving to smoke. All subjects reported that administering these two shots they could successfully refrain from smoking without any unpleasant side effects.

What is claimed is:

1. A treatment method for reducing the desire to smoke in a subject in need of such treatment essentially comprising administering 25 μL to 100 μL of a nicotine solution containing not more than 0.085 mg/mL of nicotine once through the nose.

2. The treatment method according to claim 1, wherein the treatment is applied as a nasal spray.

3. The treatment method according to claim 1, wherein the nicotine is used in a salt form.

4. The treatment method according to claim 1, wherein the nicotine solution additionally comprises inactive pharmaceutical excipients.

5. The treatment method according to claim 1, wherein the subject feels no side effects.

6. The treatment method according to claim 1, wherein the treatment is used repeatedly to enable the subject to stop smoking.
